(19) United States
(12) Patent Application Publication
(10) Pub. No.: US 2021/0151187 A1
(43) Pub. Date: May 20, 2021

(54) Data-Driven Estimation of Predictive Digital Twin Models from Medical Data

(71) Applicant: Siemens Healthcare GmbH, Erlangen (DE)

(72) Inventors: Tommaso Mansi, Plainsboro, NJ (US); Dorin Comaniciu, Princeton Junction, NJ (US)

(21) Appl. No.: 17/048,127

(22) PCT Filed: Aug. 19, 2019
(86) PCT No.: PCT/EP2019/072111

§ 371 Date: Oct. 16, 2020

Related U.S. Application Data
(60) Provisional application No. 62/721,076, filed on Aug. 22, 2018.

(51) Int. Cl. G16H 50/20; G06N 3/08; G16H 50/50; G16H 30/40

(52) U.S. Cl. CPC G16H 50/20; G16H 30/40; G16H 50/50; G06N 3/08

(57) ABSTRACT

Digital twin models of a patient, patient organ, or patient organ system from which biomarkers can be derived are used for clinical decision support. The individualization procedure also includes a predictive consideration (16) to improve the sensitivity and specificity of the digital-twin derived biomarker. In particular, during training, the predictive biomarker for which the individualized model is to be used is taken into account (16), which then accounts for the biomarker in application. The fitting (15) of the model for a specific patient accounts (16) for the prediction or model usage, resulting in estimating (14) biomarkers more optimized for the end use rather than just fit to the current baseline of the patient.

### RELATED APPLICATION

The present patent document claims the benefit of the filing date under 35 U.S.C. § 119(e) of Provisional U.S. Patent Application Ser. No. 62/721,076, filed Aug. 22, 2018, which is hereby incorporated by reference.

### BACKGROUND

The present embodiments relate to patient-specific computational models of organ function, also called digital twins. Digital twin models are used to compute advanced, multi-modal tests or scores, for clinical decision support, like non-invasive physiological measurements (e.g. fractional flow reserve (FFR), tissue stiffness or stress), markers of disease progression and prognosis, or therapy outcome prediction. For instance, individualized models of the heart have been developed to calculate biomarkers for percutaneous coronary intervention, cardiac resynchronization therapy outcome prediction or for ICD implant. Digital twins of the liver, lungs, other organs or physiological systems have been created.

Estimating a patient-specific computational model amounts to performing multi-modality data assimilation (also known as inverse modeling), eventually yielding a good fit between the model and measured patient data. In cardiac modeling for instance, medical images, 12-lead ECG, and pressure data for a patient are used to estimate values for heart shape and substrate, myocardial electrical conductivity, stiffness and stress, and/or other parameters of a multi-scale computational model. Artificial intelligence approaches, based on deep learning or deep reinforcement learning, have been developed for increased precision and robustness of the model parameters from noisy data. During model individualization, only clinical data at baseline has been used to estimate the parameters of the model.

This individualized model is then used to perform “what-if” experiments, for instance applying virtual pacing and estimate the effect on the cardiac function or other biomarker. The uncertainty of the estimated parameters may be estimated to associate the fitted computational model with a confidence score and guide the interpretation of the model predictions. Fitting a model to a patient baseline data provides a digital twin of the organ or organ system at that time only but may not be a good predictor of the organ or organ system at a later time, such as after any change due to therapy or disease progression, due to modeling assumptions, data quality, completeness, or other limiting factors. In particular, the predictive power of the model is assumed to lay exclusively in the constitutive equations of the model (either designed from experiments or learned from data). Any predictive aspect is ignored during model individualization, yielding sub-optimal results in prediction of outcome from a patient-specific model.

### SUMMARY

Systems, methods, and instructions on computer readable media are provided for estimating individualized, digital twin models of a patient, patient organ, or patient organ system, from which biomarkers can be derived for clinical decision support. The individualization procedure also includes a predictive consideration to improve the sensitivity and specificity of the digital-twin derived biomarker. In particular, during training, the predictive biomarker for which the individualized model is to be used is taken into account. The fitting of the model for a specific patient accounts for the prediction or model usage, resulting in estimating biomarkers more optimized for the end use rather than just fit to the current baseline of the patient.

In a first aspect, a method is provided for digital twin modeling in a medical system. Measurements from a patient are acquired. An estimate of a biomarker, such as clinical outcome, is determined from a model of organ function individualized to the patient by input of the measurements. The model of organ function is built based on an optimization for the clinical outcome. An image of the estimate of the clinical outcome is displayed. One or several quantitative or qualitative biomarkers to support clinical decision support may be derived.

In one embodiment, medical image data and non-medical image data are acquired for the patient.

One embodiment for individualization based on biomarkers determines values of parameters of the model of organ function. The estimate is determined using the model of organ function with the values of the parameters. The model of organ function is based on the optimization for the biomarker through selection of the model (e.g., selection of all or a sub-component of the model) of organ function from a set of multiple models (e.g., whole or sub-component models) based on clinical outcome prediction accuracy, during a training phase. Alternatively, the model of organ function is based on the optimization for the biomarker through a machine-learned model relating the measurements to the values of the parameters. The machine-learned model was trained with a loss function including a first term for a difference of training measures to model output and a second term for a difference from training biomarkers to model biomarker. In that way, the mapping between clinical data and model parameters takes into account the biomarker to predict, thus reducing the manifold of potential parameter values to the space relevant to the user. Various machine-learned models may be used, such as a neural network, for instance, an encoder, a decoder, and an estimation network receiving values of bottleneck features between the encoder and the decoder.

Another embodiment for individualization based on biomarker determines the estimate of the biomarker (e.g., clinical outcome) from input of the measurements to a machine learned model, which outputs the estimate. The machine-learned model was trained based on the optimization for biomarker. For example, the machine-learned model was trained with a loss function with a first term for a distance between a constitutive model and a model output of the machine-learned model and a second term for a distance between biomarkers. As another example, the machine-learned model was trained as a forward model. The forward model may have been pre-trained based on output from a generative computational model.

In a second aspect, a medical system is provided for digital twin modeling. A medical imager is configured to scan a patient. An image processor is configured to predict a biomarker with a digital twin model of a physiological system of the patient. The digital twin model is individualized to the patient based on biomarker prediction and data from the scan and/or other data sources. A display is configured to display the clinical outcome.

In one embodiment, the digital twin model is individualized based on the biomarker prediction by selection from a group of models during training where the selection was based on comparison using a plurality of samples of scan data and biomarkers. In another embodiment, the digital twin model is individualized based on the biomarker prediction by output of values of parameters of the digital twin model by a machine-learned model in response to input of the data from the scan with or without other clinical data. The machine-learned model was trained using a loss function including a distance in predicted biomarker. In yet another embodiment, the digital twin model directly returns the new biomarker, the digital twin model being a machine-learned model trained using samples of scan data and outcomes. For example, the machine-learned model was trained with a loss function having a first term based on distance from a constitutive model and a second term based on distance in predicted biomarker. As another example, the machine-learned model was trained as a forward model from the samples and therapy parameters.

In a third aspect, a method is provided for organ modeling to a patient in a medical system. An organ of a patient is modeled from measurements of the patient. The response to therapy, disease progression, and/or prognosis is accounted for in the modeling of the organ of the patient. An estimate of patient outcome is generated using the modeling.

In one embodiment, to account for the response, a first model is selected from a plurality of models. The selection is based on comparison of accuracy in prediction of the response to the therapy, disease progression, and/or prognosis of the models using testing data. In another embodiment, to account for the response, values of parameters of a model used in the modeling are estimated by a machine-learned model. The machine-learned model was trained with a loss function including a loss term for the response to the therapy, disease progression, and/or prognosis. In yet another embodiment, to account for the response, the estimate is generated with a machine-learned model. The machine-learned model was trained with training data including the patient outcome.

Any one or more of the aspects or concepts described above may be used alone or in combination. The aspects or concepts described for one embodiment may be used in other embodiments or aspects. The aspects or concepts described for a method or system may be used in others of a system, method, or non-transitory computer readable storage medium.

These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 is a flow chart diagram of one embodiment of a method for individualizing a digital twin model based on clinical outcome;

FIG. 2 illustrates a progression of data for patient-specific modeling using outcome;

FIG. 3 shows an example network for estimating values of parameters of a model for individualization;

FIG. 4 shows another example network for estimating values of parameters of a model of individualization; and

FIG. 5 is a block diagram of one embodiment of a medical system for organ modeling with a patient-specific fit using outcome.

### DETAILED DESCRIPTION OF EMBODIMENTS

Digital twins of patients' organ, organ systems or body are poised to provide new clinical biomarkers to support clinical decisions. The biomarker is an non-measured indicator of the severity or presence of some disease state, such as an indicator of a particular disease state or some other physiological state. The biomarker may be a clinical outcome, which example is used herein. The biomarker may be a risk score, disease progression level or state, or an estimate of operation (e.g., fractional flow reserve). The ability to estimate non-invasively physiological parameters, to perform ‘what-if” scenarios and to compute new values for biomarkers will be part of next generation clinical decision support systems.

A major limitation of existing digital twin methods is that the predictive power of the system is assumed to lay exclusively in the constitutive equations that describe the digital twin model, i.e. in their ability to replicate the biophysiological phenomena. The clinical biomarkers to predict, like for new risk scores or therapy outcome, have not been explicitly used to build these models. Clinical outcome (e.g., disease progression or therapy outcome), risks scores (e.g. risk of developing a disease or of non-response) or non-invasive physiological parameters (e.g. tissue stiffness or flow pressure) are not used when creating the individualized model. Any predictive aspect is ignored during model design, while only clinical data at baseline is used to estimate the parameters of the model, yielding sub-optimal results.

Predictive digital twin models are estimated from medical data using data-driven techniques that consider the clinical biomarker of interest during the modeling process. By combining computational modeling and artificial intelligence (neural networks), generative and predictive computational models of organs and organ systems that optimize their accuracy and predictive power by taking into account outcome data are provided. A training dataset that contains baseline data and clinical biomarkers of interest (e.g. outcome data) is used to optimize the digital twin estimation process. In one approach, the best model from among a set of available models is selected and/or tuned such that baseline and clinical biomarkers are best captured in the training set. For application, the selected model is used. In another approach, a mapping between (1) baseline and clinical biomarker and (2) digital twin model parameters is provided such that model goodness of fit at baseline and of the clinical biomarker is optimal. The inverse problem is learned such that estimated parameters lay in the manifold that best predict the clinical biomarkers of interest. In yet another approach, a new computational model is directly learned from baseline and the clinical biomarkers to predict such that model goodness of fit at baseline and clinical biomarkers is optimal. In a further approach, the input data is compressed and/or integrating into a task specific fingerprint that summarizes the input data while exhibiting outcome prediction capabilities.

The digital twin model is a model of an organ, organ system, human body, or another physiological system of the patient. The digital twin model is a computational model that is personalized, such that it captures a patient's physiology like a virtual twin system. The model may be multi-scale, multi-physics, or learned from a large database, reflecting various characteristics of the physiological system, such as elasticity, thermal conduction, electrical, structural, and/or operation. Alternatively, the model represents one characteristic.

The digital twin model, after personalization to measurements from the patient, is used to predict clinical outcome, compute new biomarkers to support clinical decision. The model may provide a measurement of performance of the modeled system, such as a fractional flow reserve. This performance may be used to indicate an outcome, such as mapping the fractional flow reserve to success or not of therapy applied to the system of the patient. New values of clinical biomarkers for disease progression, and/or disease or event risks (e.g., survival or recurrence after a given number of years) can be derived.

In the examples below, a cardiac use case is used, without loss of generality. Other systems will work as well, like for instance liver, lung, musculoskeletal, cancer, etc. For the cardiac use case, the digital twin model represents one or more characteristics of the heart or other part of the cardiac system. For example, a virtual heart model is used to compute biomarkers, such as outcomes, of cardiac arrhythmias. As another example, a virtual angiogram model of the heart for predicting fractional flow reserve is used. In other embodiments, the modeling is of other organs, such as a virtual model of the liver or lungs. The modeling of any physiological system is optimized to provide the best clinical biomarkers, in terms of sensitivity, specificity or other relevant metrics, for a specific patient. In the examples below, clinical outcome is used as the biomarker, but other biomarkers may be used.

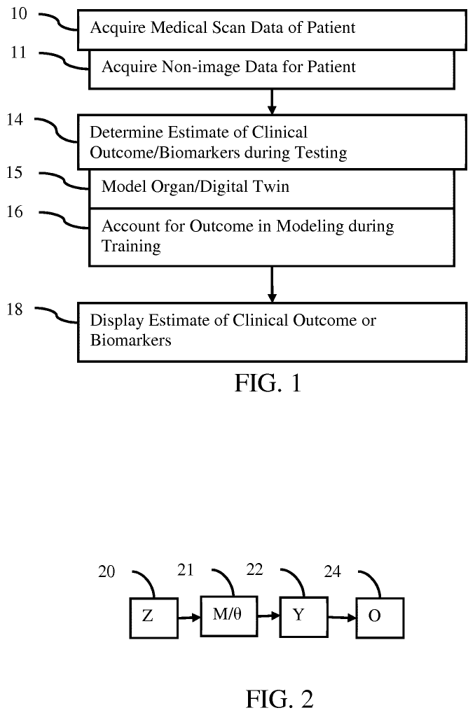

FIG. 1 is a flow chart diagram of one embodiment of a method for individualized digital twin modeling in a medical system. The model includes outcome as the clinical biomarker consideration. In other words, the ability of the personalized model to accurately model predictive clinical biomarkers, such as outcome, is accounted for in digital twin modeling.

The modeling for a given patient (i.e., during application) includes biomarker consideration through the use of biomarkers for other patients during training. The training accounts for the biomarkers to provide a model and/or fitting better able to predict biomarkers. This model or fitting is applied for a given patient, thus accounting for the unknown value of the biomarker in the patient.

The method of FIG. 1 is performed in the order shown (e.g., top to bottom or numerical), but other orders may be used. For example, acts 15 and 16 are performed simultaneously or as part of fitting the model to the data for the patient.

Additional, different or fewer acts may be provided. For example, one of acts 10 or 11 are not performed. As another example, acts for fitting are performed.

The method is performed by one or several medical scanners, one or several non-imaging scanners (e.g. lab diagnostics, ECG, wearable, etc.), a workstation, a server (on premise or in the cloud), or a computer. The scanner or memory are used to acquire data for a patient. An image processor, such as an image processor of the scanner or a separate computer (on-premise or in the cloud), determines an estimate of clinical biomarker of interest using the digital twin model. The image processor uses a display screen or printer. A physician may use the output information to make a clinical decision for the patient (e.g. treat/no treat, perform this or that intervention, etc.).

In one embodiment, a healthy patient wears a wearable sensor, such as a pulse and pressure sensor. The modeling may be based on this baseline sensor data for providing the estimated biomarker to the patient. In alternative embodiments, the modeling is performed for a non-healthy patient to provide the predicted biomarker to the patient and/or physician

In act 10, an image processor acquires one or more medical scans of a patient. The scan data from the scan of the patient is acquired from a medical scanner, such as a computed tomography (CT) scanner. The computed tomography scanner scans a patient with x-rays using an x-ray source and detector mounted to a gantry on opposite sides of a patient. A magnetic resonance (MR), positron emission tomography, single photon emission computed tomography, and/or ultrasound scanner may be used instead of or in addition to the CT scanner. In alternative embodiments, scan data from a previous scan of the patient is acquired from a memory or transfer over a computer network.

The input is one or several medical image, such as scan data. The scan data represents an area or volume of the patient. For example, the scan data represents a three-dimensional distribution of locations or voxels in a volume of the patient. The distribution of locations may be in a Cartesian coordinate system or uniform grid. Alternatively, a non-uniform grid, polar or cylindrical coordinate system, or any other coordinate system, is used. For representing a volume, a scalar or vector-based value is provided for each voxel representing the volume.

The scan data may be pre-processed before being used to fit the model to the patient. Pre-processing may include segmentation, filtering, normalization, scaling, or another image processing. For example, one or more tumor volumes (e.g., gross tumor volume) or regions including the tumor with or without non-tumor tissue are segmented. The segmentation may be by manual delineation or automatically by the image processor. The scan data to be input represents just the segmented region or separate inputs are provided for the segmented region and the entire scan volume. The scan data (e.g., image data) with and/or without pre-processing is used to estimate the digital twin model, from which clinical biomarkers are derived (e.g. to predict outcomes).

Non-image data may be input instead or in addition to scan data. In act 11, the image processor acquires non-image data. The non-image data is from sensors, the computerized patient medical record, manual input, pathology database, laboratory database, wearables, and/or other source. The non-image data represents one or more attributes of the patient, such as family history, medications taken, temperature, body-mass index, pressure, pulse, and/or other information. For example, genomic, clinical, measurement, molecular, and/or family history data of the patient are acquired from memory, transform, data mining, and/or manual input. In another example, proposed therapy settings are acquired, such as a course of therapy including a sequence of therapy events, the power for each event, the duration of each event, device to be used, implantation process, implantation location, and/or the region of application.

In the cardiac system or heart modeling, the non-image data may include cardiac electrocardiogram (ECG) data, blood pressure, blood characteristics from laboratory tests, wearable signals, family history, genetic information, and/or other information. The image data may be ultrasound, x-ray, MR, CT, and/or other scan data representing spatial distribution of tissue, blood, contrast agent, and/or function in the heart or vessels.

In act 14, an image processor uses the digital twin (e.g., model of organ function) to determine an estimate of clinical biomarkers of interests, such as clinical outcome. For example, a multi-scale model, such as a model of the heart including anatomy, hemodynamics, biomechanics, and electrophysiology, as fit to the patient is used to estimate clinical biomarkers, such as outcome, for decision support.

FIG. 2 shows a model of the information used in digital twin modeling to determine the clinical biomarker. Measurements Z 20 for a given patient are used to fit a model M 21. The fit solves for values of one or more parameters θ of the model M 21 so that the output values Y 22 match the measurements Z 20. Once personalized to the patient, the model 21 and corresponding values of the parameters θ are used to calculate new clinical biomarkers by for instance determining operation of the modeled organ or another physiological system after change. One or more characteristics of the model M 21 are altered to reflect the change, such as altering one or more values of the model parameters θ. The outcome O 24 is estimated directly by the model M 21 (e.g., the outputs Y 22 represent the outcome O 24). Alternatively, the model 21 outputs one or more values Y 22 for operation of the organ due to the change, which outputs are then used to determine the outcome 24. Alternatively, the parameters θ themselves can be considered as clinical biomarker.

The image processor generates a clinical biomarker, such as a prediction of outcome from therapy or disease progression, for the patient. For example, a risk of reoccurrence or response to device therapy is predicted. In other embodiments, the model is used to estimate a prognosis of a disease or other future operation or event as the clinical outcome.

In the example of cardiac electrophysiology, the model outputs the QRS duration, QT duration, and electrical axis, among other parameters. By altering a parameter of the model, such as local conduction velocities or by adding virtual stimuli, the effects of therapy may be estimated. The QRS duration, QT duration, and electrical axis resulting from the alteration indicate the outcome from the therapy. Other outcome, such as a binary ceasing of irregular heartbeat, or risk scores, such as probability of long-term response to a treatment, may be derived from the output of the model after alteration.

Any of various types of models may be used. For example, a computational model or model represented by equations or relationships is used. A constitutive model may be used. A machine-learned model may be used. For example, a neural network is trained to generate the model output given the measurements as input.

The model may include any number of scales or details. For example, any level of detail of underlying constitutional laws, e.g. use more or less sophisticated cellular models, may be used. Any level of details of the anatomical model, e.g. add or remove healing tissue and/or detailed conduction pathway, may be used. Different constitutive laws may be used, such that different functions capture baseline and outcome in different ways or to different extents. For example, different equations of cardiac electrophysiology may be used, either derived from wet lab experiments or directly learned from the data.

To estimate the clinical biomarker for a given patient, the image processor fits the model to the patient in act 15. A digital twin model to the organ or another physiological system is created. The measurements for the patient, such as the image and non-image data, are used to solve for the values of the parameters of the model. The values of parameters of the model that result in the model output most closely matching the measurements or values derived from the measurements is found. This personalized model is then used to generate the estimate of the clinical biomarker, for instance a parameter related to patient physiology (e.g. pressure) or a risk score associated to a therapy outcome specific to that patient.

An organ or other system of a patient is modeled. The model is individualized or personalized to the patient from measurements of the patient. Let y=f(θ) be the computational model, where y is a set of output parameters, θ the model parameters and f the model function. For instance, f can be a cardiac electrophysiology model controlled by θ=(c_{LV}, c_{RV}, c_{Myo}) the conduction velocities of the left ventricle, right ventricle and myocardium respectively, and y=(QRSd, QTd, EA) the QRS duration, QT duration and electrical axis. f can represent any other computational model, parameterized by any other parameters.

Traditionally, model individualization estimates θ such that the distance between y and measured values z, denoted D(y,z), is minimized. The baseline measurements (i.e., current measurements or current and past measurements) are used to estimate the digital twin model. Minimizing D(y,z) does not guarantee that the model f is predictive. In other words, nothing guarantees that if θ varies for instance, the new output of the model still matches the clinical observations when similar changes are observed in the patient. This generalization is even more difficult to obtain accurately if one aims to predict disease progression or the effects of a therapy.

In act 16, the image processor individualizes the modeling of act 15 to account for response to therapy, disease progression, and/or prognosis in the modeling of the organ of the patient. At test time, i.e. for application to a new patient, the clinical biomarker, such as outcome, is not known so cannot be used to fit the model. The know biomarker information from other patients is used when creating the model, or estimating the mapping between parameters and measurements, or by learning the model, at training time. The training accounts for outcome so that the resulting model information accounts for the biomarker at test time.

For example, a multi-scale model of organ function is optimized to better capture the clinical biomarker, such as outcome, after personalization. The outcome after time or change for which the personalized model is to be used to predict is used in the modeling. The outcome may be the output of the model after alteration of the model or the estimate determined from the output of the model. The model is personalized in a way that accounts for performance of the fit model in predicting outcome after change or alteration. The values of parameters of the multi-scale model of organ function are determined in a way that accounts for clinical outcome, and the estimate of act 14 uses the multi-scale model of organ function with the values of the parameters determined using outcome.

Let g be a function that models a disease or therapy process (e.g. ablation or device therapy, disease progression, or time). g acts on the organ model f, such that y=g(f(θ), γ), where y is the set of parameters associated to the therapy or disease process. The goal is to estimate f and θ to maximize the predictive performance of the model given g. Estimating f means selecting the model details that are necessary for a good prediction. Estimating θ means finding the parameters such that baseline organ function and its change under disease progression, therapy, or other change indicating clinical outcome is captured.

The clinical biomarker, such as outcome, may be accounted for in the individualization of the model and or the model itself in various ways. In a first way of accounting for clinical biomarker or other predictive capability of the model, model selection is used. The model of organ function (e.g., multi-scale model) is based on optimization for the clinical outcome through selection of the model of organ function from a set of multiple models. The selection is based on clinical outcome prediction accuracy in a training set.

Any number of models may be available. The different models may be tested for ability to predict the outcome once fit to patients. Models can also be extended with additional features to improve prediction accuracy. One model is selected from a plurality of models based on comparison of accuracy in prediction of the response to therapy, disease progression, prognosis, and/or another clinical outcome of the models using testing data. The selected model is the model optimized for prediction or outcome and is used to fit to the patient for determining the estimate of clinical outcome. In this approach, the model f is adapted to maximize prediction accuracy through selection. Parameter estimation for fitting is not changed as the parameter estimation is assumed to provide an accurate estimate of the model parameters from the available data.

For selection in the outcome example, one or more samples that include measurements and outcome for one or more changes are provided. The different models are used to model each sample and predict outcome. The predicted or estimated outcomes are compared to the provided outcome. The accuracy may be measured in various ways, such as an average difference or weighted summation of difference across samples.

In one embodiment, the different models are formed from one starting model. Given a complete dataset including baseline (e.g., measurements) and outcome data, the model is progressively adapted such that the entire pipeline “model fitting→parameter estimation→virtual disease progression/intervention→outcome prediction” provides the best performance in terms of specificity, sensitivity, and/or another statistical metric. After each fit, the pipeline models a change (e.g., progression or intervention represented by a change in one or more values of the parameters of the model) and provides for estimation the clinical outcome from the model as changed. The modified model can then be used to new patients, during “testing”.

Different alterations of the model may be used to find the model resulting in the best performance. Model adaptation includes, but is not limited to: (1) change in level of details of underlying constitutional laws, e.g. use more or less sophisticated cellular models, (2) change the level of details of the anatomical model, e.g. add or remove healing tissue or detailed conduction pathway, (3) adapt constitutive laws such that they better capture baseline and outcome, like for instance modifying the equations of cardiac electrophysiology to better consider the effect of non-natural pacing, and/or (4) another modification of the model prior to fitting. Model selection is done on a training and validation dataset. Final evaluation of the model performance may be achieved on an independent testing dataset.

In other embodiments, the different models are provided in a database. The accuracy of predictions of the models as fitted to each sample is used, in part, to select the model to be used for a patient or application. By using this selected model, the estimation of act 14 and modeling of act 15 accounts for the outcome in act 16.

In a second way of accounting for clinical biomarkers, outcome or other predictive capability of the model, a machine-learned model is used to generate the values of the parameters of the digital twin model. The machine-learned model performs the fitting. The model of organ function is based on the optimization for the clinical outcome through a machine-learned model relating the measurements for the patient to the values of the model parameters. The machine-learned model provides values that account for the clinical biomarker of interest. For example, the machine-learned model was trained with a loss function including a first term for a difference of training measures to model output and a second term for a difference from training outcomes to model-based outcome.

Multi-task inverse modeling is provided for outcome prediction. Manifolds of parameters may yield similar observations (e.g. ejection fraction, stroke volume). The question is then which set of parameters provides both fidelity at baseline and predictive accuracy. Machine learning may be used to learn the inverse mapping function with integrated outcome data during the learning process. The machine learning includes one task for estimating values of parameters of the model (i.e., personalizing) and another task for accuracy in predicting the outcome due to change. The tasks are reflected in the loss function for machine learning.

The goal is to find the model parameters θ such that the output parameters y match the baseline measurements z and the predicted outcome g(f(θ), γ) is as accurate as possible. Outcome measurements are provided in the training set. The goal is therefore to learn with a loss function h such that D(f(h(z)), z)+λ D(g(f(h(z)), γ) is minimized, where λ is a weight parameter. In this loss function, a weighted sum of two terms—loss or difference for the baseline and loss or difference for predictive accuracy in outcome—is used. Other loss functions for multi-task machine learning may be used. During testing, h(z) is calculated to get the model parameters θ that are associated with the highest prediction accuracy.

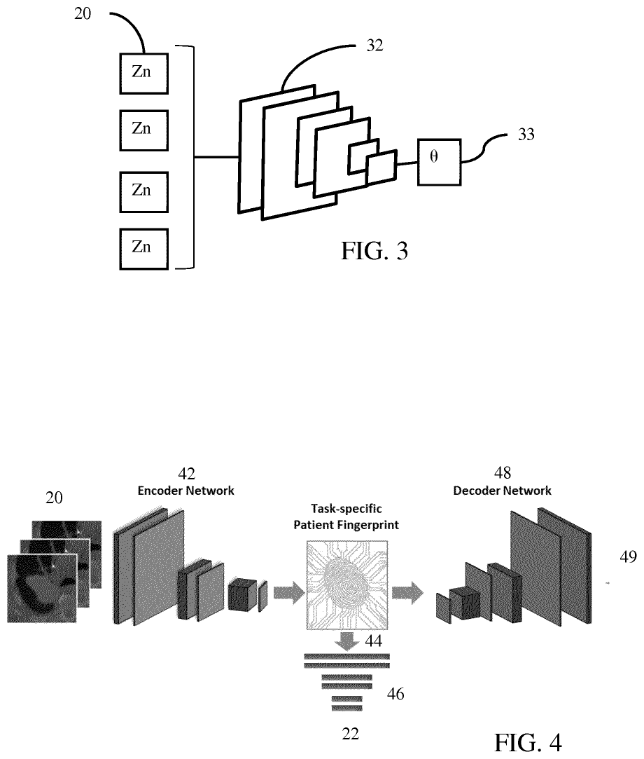

Various approaches may be used to estimate an approximation of the function h. In one embodiment, a neural network is trained as a machine-learned model to operate as a general function approximator. FIG. 3 shows a possible neural network architecture. The inputs are the set of measurements Zₙ 20. The outputs are the estimated parameters θ 33. Any number of layers and corresponding network structure may be used for the network 32, such as a dense net or fully convolutional network.

The loss function is D(f(h(z)), z)+λ D(g(f(h(z)), γ), whose derivative may be computed using numerical approaches for machine learning. Alternatively, an approximation of the model f may be employed, for instance using polynomial chaos, to speed up the training process by removing the need of performing forward model computations to calculate the gradients. Any other loss function could also be used, provided the loss function captures both baseline and clinical biomarker prediction.

In this first embodiment, the input parameters Zₙ 20 are discrete values. Other networks or processing may be used to determine the discrete values from the image and/or non-image data for the patient. Other machine-learned models may be used, such as models learned through manifold learning or regression.

FIG. 4 shows an embodiment for another approach to approximate the function h. In this example, the raw image and/or non-image data is input directly by using a U-Net, image-to-image, or other generative architecture. The network architecture for the machine learning model includes an encoder 42, bottleneck 44, and decoder 46 (e.g., together forming an autoencoder). For application, the decoder 46 may not be used. For generating the values of the model parameters for personalization of the digital twin model, the network architecture includes a convolutional neural network or other estimator network 46 connected to receive output of the encoder 42 or the bottleneck 44 as input.

The encoder 42 and decoder 48 are trained for one task, such as to generate an output 49 representing the input data 20 or information shown in the input data 20. The estimation network 46 is trained for another task, such as to generate the values of the parameters of the organ model from the values of features at the bottleneck 44. The embodiment of FIG. 4 leverages other image and/or signal features to estimate the model parameters, compared to using discrete values directly. The multi-task architecture also ensures that the network learns an encoding that is relevant for model prediction through use of the loss function with multiple terms including a term accounting for outcome prediction.

Other network architectures may be used to train a machine-learned model to generate the values of the parameters for personalizing the digital twin model while accounting for optimum outcome prediction. By using loss or task for outcome, the machine-learned model is trained to output values that result in more accurate outcome prediction than using only loss based on the baseline (i.e., not accounting for outcome).

For training the machine-learned network, the machine learning network arrangement is defined. The definition is by configuration or programming of the learning. The number of layers or units, type of learning, order of layers, connections, and other characteristics of the network are controlled by the programmer or user. In other embodiments, one or more aspects of the architecture (e.g., number of nodes, number of layers or units, or connections) are defined and selected by the machine during the learning.

The machine (e.g., processor, computer, workstation, or server) machine trains the defined network (e.g., the defined multi-task generator). The network is trained to generate outputs for one or more tasks, such as multiple tasks. The generator and any discriminators are trained by machine learning. Based on the architecture, the generator is trained to generate output.

The training data includes many samples (e.g., hundreds or thousands) of input data 20 (e.g., image and/or non-image data) and ground truths (e.g., values for parameters of the model, outcome, and/or data for other tasks). The network is trained to output based on the assigned ground truths for the input samples.

For training any of the networks, various optimizers may be used, such as Adadelta, SGD, RMSprop, or Adam. The weights of the network are randomly initialized, but another initialization may be used. End-to-end training is performed, but one or more features may be set. The network for one task may be initially trained alone, and then used for further training of that network for the one task and a further network for the other task. Separate losses may be provided for each task. Joint training may be used. Any multi-task training may be performed. Batch normalization, dropout, and/or data augmentation are not used but may be (e.g., using batch normalization and dropout). During the optimization, the different distinguishing features are learned. The features providing an indication of outcome and indication of values for parameters to personalize the model are learned.

The optimizer minimizes an error, difference, or loss, such as the Mean Squared Error (MSE), Huber loss, L1 loss, or L2 loss. The same or different loss may be used for each task. In one embodiment, the machine training uses a combination of losses from the different tasks.

Once trained, the machine-learned model outputs patient-specific, predictive values for model parameters (i.e., θ) in response to input of measurements (e.g., image and/or non-image data for the patient). The values output are ones more likely than other values that could result from personalization to accurately predict outcome in response to change in the model. By using the machine-learned model that was trained with a loss function including a loss term for the response to the therapy, disease progression, and/or prognosis, the generated values for personalization may accurately predict outcome.

In a third way of accounting for clinical outcome or other predictive biomarker of the model, a machine-learned model is used as the digital twin (e.g., multi-scale model of an organ). The machine-learned model implements the forward modeling of the digital twin. To personalize while accounting for outcome, the machine-learned model is trained using outcome. The machine-learned model is trained to determine the estimate of the clinical outcome or model output from input of the measurements. The machine-learned model was previously trained based on the optimization for clinical outcome. The machine-learned model is a data-driven computational model that favors outcome prediction accuracy. The machine-learned model is trained as a predictive forward model f(θ) directly. The training data includes the clinical outcome or outcome used to derive the clinical outcome. The output of the forward model representing change from the baseline is included in the training data.

Various approaches to directly learn a forward model that is as accurate as possible in terms of clinical biomarker may be used. In one approach, data-driven constitutive modeling is used. The machine-learned model was trained with a loss function with a first term for a distance between a constitutive model and a model output of the machine-learned model and a second term for a distance between outcomes. The constitutive model associated to the physiological system of interest (e.g. action potential model or excitation-contraction model) may be injected into the digital twin computational framework (e.g. finite element or lattice Boltzmann solvers) for multi-scale integration through machine training. Several constitutive models could yield the same observable organ function, but some may provide more accurate predictions than others for a specific clinical question. Outcome data is used to train a constitutive model that is optimal for the prediction task of interest.

A validated and accepted constitutive model c is used in training, such as a model of cardiac action potentials. c captures observed physiology under varying healthy conditions, which often does not include disease processes or therapy. c is to be replaced by a machine-learned model c⁺ learned from data that approximates c while providing higher prediction accuracy. Machine learning infers a generative model that can capture the behavior of c without having to solve all the differential equations of c. Manifold learning and/or regression analysis may be used. In one embodiment, a generative neural network based on variational auto-encoders is used.

The model c⁺ is learned by adding the prediction task to the loss function. For example, the loss function is represented as: D(c, c⁺)+λ D(g(f(θ), γ). In this loss function, the model individualization algorithm is fixed. Both individualization and outcome prediction may be combined by following an alternate optimization procedure (e.g. ADMM). By determining loss with a term relating a difference between the validated constitutive model as personalized and the personalized machine-learned model, the accuracy in values of the parameters of the model are optimized in training. By including loss with a term relating a difference in outcome prediction between a ground truth and the personalized machine-learned mode, the accuracy in outcome prediction of the model is optimized in training.

In another approach, the forward model is learned directly. The complete forward model f(θe) may be learned. The inputs are the clinical measurements at baseline and therapy parameters describing the change to be made, and the output is the outcome parameter of interest or an output from which outcome is derived. The input parameters are clinical measurements at baseline, for instance discrete values like lab tests, ECG parameters, clinical data, or complete raw signals like images or ECG traces. A neural network, such as the network of FIG. 3, or another machine-learning model may be used. The therapy parameters are used as inputs to the network with the image and non-image data. The output of the machine-learned model may be a classification (i.e., responder or non-responder) or detailed outcome data like changes in cardiac function for instance (i.e., a regression task).

One challenge for training such a neural network is the need of a large amount of data that includes disease and/or therapy variability. To cope with this challenge, a generative computational model of the organ function may be used to simulate various disease and/or therapy scenarios and hence generate millions of synthetic data as training data. This synthetic data is used to pre-train the network. The pre-trained machine-learned model is then further trained on samples or training data from actual patients. To maximize the efficacy of the approach, pre-training may be done on a network that was first optimized for the prediction task following the model selection or model parameter estimation approaches.

In one example using either forward model as trained, for cardiac resynchronization therapy, a large database of annotated data with outcomes is used to train a machine-learned model that, given baseline imaging parameters (e.g., from MR or ultrasound, like hemodynamics parameters, scar burden, ECGs, etc.) and therapy options (e.g., device parameters and/or lead location) predicts the outcome metric of interest (e.g., acute QRS shortening or max dp/dt, long-term hemodynamics changes like changes in end systolic volume or ejection fraction, etc.). The approach could be applied to other applications, for instance risk of sudden cardiac death, plaque rupture, stroke, etc.

In act 18, the image processor causes display of an image of the estimate of the biomarker, such as clinical outcome, or a value derived therefrom. The estimate may be an output from the model, such as the QRS duration, QT duration, and/or electrical axis, after a change. The estimate may be derived from the output of the model, such as survival, event risk, and/or disease risk derived from output of tumor size, wall thickness, or elasticity.

In one embodiment, the outcome is a likelihood of therapy failure or success. The success may be based on lack of increase in size or tumor being gone. The success may be a measure at a given time after therapy. The outcome may be for recurrence. Any measure of therapy or clinical outcome given a change in the modeled organ may be used.

By predicting the outcome or other biomarker, the physician can determine whether a given therapy may be appropriate for a given patient, whether treatment is needed, and/or use disease progression or prognosis information in decision making for the patient. The prediction may be for outcomes for more than one type of therapy so that the physician may select the more likely to be successful therapy.

In one embodiment, the outcome is predicted as a survival. Rather than a binary prediction, the prediction may be of a continuous variable, such as probability of survival as a function of time. The survival may be a time-to-event (e.g., 28 months). The time-to-event may be a time between treatment and recurrence and/or a time until death.

In other embodiments, disease progression, stage, estimate of operation, or other biomarker is output. The image includes information for one or more biomarkers predicted from the modeling.

The image includes alphanumeric text or a graph indicating the estimated clinical outcome. Other information may be included, such as a medical image (e.g., CT, MR, or ultrasound). The estimated clinical outcome may be an annotation on an image representing spatial distribution of anatomy and/or function in medical imaging. The estimated clinical outcome may instead be displayed in a chart, as part of a medical record, or in a radiology or other report.

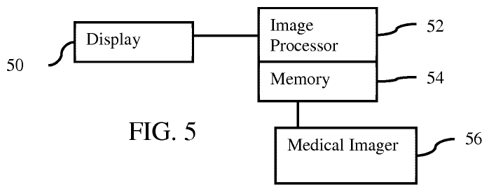

FIG. 5 shows a medical imaging system for individualized digital twin modeling. A physiological system (e.g., organ or organ system) is modeled. The model is fit to a patient. The fitting includes consideration for or accounts for ability to predict one or more biomarkers in response to a change in the model, such as to simulate therapy, disease progression, and/or passage of time. The system implements the method of FIG. 1 or a different method.

The medical imaging system includes the display 50, memory 54, and image processor 52. The display 50, image processor 52, and memory 54 may be part of the medical imager 56, a computer, server, workstation, or other system for image processing medical images from a scan of a patient. For example, the modeling and/or prediction are performed by a server or other cloud-based computer. A workstation or computer without the medical imager 56 may be used as the medical imaging system.

Additional, different, or fewer components may be provided. For example, a computer network is included for remote prediction based on locally captured scan data. As another example, a user input device (e.g., keyboard, buttons, sliders, dials, trackball, mouse, or other device) is provided for user interaction. In yet another example, a remote database, such as for storing laboratory results, a computerized patient medical record, or other image or non-image data, is provided. In other examples, measurement devices, such as pressure monitor, ECG, pulse monitory, and/or oxygen content monitor, are provided.

The medical imager 56 is a computed tomography, magnetic resonance, ultrasound, x-ray, fluoroscopy, angiogram, positron emission tomography, single photon emission computed tomography scanner, and/or another modality scanner. For example, the medical imager 56 is a computed tomography system having an x-ray source and detector connected to a moveable gantry on opposite sides of a patient bed.

The medical imager 56 is configured by settings to scan a patient. The medical imager 56 is setup to perform a scan for the given clinical problem, such as a lung or heart scan. The scan results in scan or image data that may be processed to generate an image of the interior of the patient on the display 50. The scan or image data may represent a three-dimensional distribution of locations (e.g., voxels) in a volume in the patient, a two-dimensional distribution of locations in an area in the patient, or a one-dimensional distribution of locations along a line in the patient.

The medical imager 56 provides image data. Multiple different modalities of medical imagers 56 may be used, such as providing multiple sets of image data for a same patient. Non-image data may be provided in the memory 54, by transfer from a database over a computer network, user entry on an input device, and/or connection with a measurement device.

The image processor 52 is a control processor, general processor, digital signal processor, three-dimensional data processor, graphics processing unit, application specific integrated circuit, field programmable gate array, artificial intelligence processor or accelerator, digital circuit, analog circuit, combinations thereof, or other now known or later developed device for processing medical image and/or non-image data. The image processor 52 is a single device, a plurality of devices, or a network. For more than one device, parallel or sequential division of processing may be used. Different devices making up the image processor 52 may perform different functions. In one embodiment, the image processor 52 is a control processor or other processor of a medical diagnostic imaging system, such as the medical imager 56. The image processor 52 operates pursuant to stored instructions, hardware, and/or firmware to perform various acts described herein.

The image processor 52 is configured to predict a clinical outcome or other biomarker with a digital twin model of a physiological system of the patient. For example, the outcome from therapy is predicted from a digital twin model of the heart. The model is fit to or personalized to the patient using the imagine and/or non-imaging data. Once fit, the model is altered to simulate application of the therapy or passage of time. The fit model as altered is then used to predict the outcome of the therapy, such as predicting flow (e.g., fractional flow reserve) and/or predicting survival resulting from a change in flow. Alternatively, the fit and alteration are combined so that the model outputs based on input of the measurements and alteration parameters.

To better operate in prediction of outcome or other biomarker, the digital twin model is individualized to the patient based on biomarker prediction and data from the scan. Biomarker prediction performance is included in fitting the model. The biomarker prediction is included by training using known biomarkers so that the biomarker prediction in a current application with an unknown biomarker value operates better.

In one embodiment, the digital twin model is individualized based on the biomarker prediction by selection of the model from a group of models where the selection was based on comparison using a plurality of samples of scan data and biomarkers. Various samples of model inputs, model outputs and biomarker with or without progression or therapy parameters are used to test different models to identify the model that performs best or sufficiently in biomarker prediction. By fitting the selected model, the individualization for the current patient with the unknown value of the biomarker is optimized for outcome prediction.

In another embodiment, the digital twin model is individualized based on the biomarker prediction by output of values of parameters of the digital twin model by a machine-learned model in response to input of the data from the scan. To account for the biomarker, the machine-learned model was trained using a loss function including a distance in predicted biomarker. The training data includes biomarker, which may be used for loss based on biomarker in training. The resulting machine-learned model provides values for the model parameters of the digital twin model that both fit to the patient and optimize for biomarker prediction in the current application.

In yet another embodiment, the digital twin model is individualized based on the biomarker prediction by output of the clinical biomarker by the digital twin model where the digital twin model is a machine-learned model trained using samples of scan data and values of biomarkers. Rather than training the machine-learned model to output the values of the model parameters for fitting to the patient based, in part, on the biomarker, the machine-learned model is trained to be or replace the digital twin model. In one embodiment, the machine-learned model was trained with a loss function having a first term based on distance from a constitutive model and a second term based on distance in predicted biomarker. The constitutive model is used as a starting point, allowing training in machine learning to provide output like the constitutive model based on the difference from the constitutive model. The output regression or classification is altered to include consideration of biomarker using an outcome loss. In another embodiment, the machine-learned model was trained as a forward model from the samples and therapy parameters. Any parameterization of alteration for which biomarker is to be predicted is used as an input. The machine training learns to output based on the inputs of the image data, non-image data, and change (e.g., values for therapy parameters). The biomarker may be used in a loss in the training.

The image processor 52 applies the individualized model. Any resulting output due to simulated alteration or passage of time is used as or to derive the estimate of clinical biomarker.

The image processor 52 is configured to generate an image. An image showing the predicted biomarker is generated. The biomarker may be displayed with an image of the interior of the patient, such as a computed tomography image. The predicted biomarker is displayed for decision support.

The display 50 is a CRT, LCD, projector, plasma, printer, tablet, smart phone or other now known or later developed display device for displaying the output, such as an image with a prediction of clinical biomarker.

The scan data, training data, network definition, features, machine-learned network, non-image data, biomarker, and/or other information are stored in a non-transitory computer readable memory, such as the memory 54. The memory 54 is an external storage device, RAM, ROM, database, and/or a local memory (e.g., solid state drive or hard drive). The same or different non-transitory computer readable media may be used for the instructions and other data. The memory 54 may be implemented using a database management system (DBMS) and residing on a memory, such as a hard disk, RAM, or removable media. Alternatively, the memory 54 is internal to the processor 52 (e.g. cache).

The instructions for implementing the training or application processes, the methods, and/or the techniques discussed herein are provided on non-transitory computer-readable storage media or memories, such as a cache, buffer, RAM, removable media, hard drive or other computer readable storage media (e.g., the memory 54). Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination.

In one embodiment, the instructions are stored on a removable media device for reading by local or remote systems. In other embodiments, the instructions are stored in a remote location for transfer through a computer network. In yet other embodiments, the instructions are stored within a given computer, CPU, GPU or system. Because some of the constituent system components and method steps depicted in the accompanying figures may be implemented in software, the actual connections between the system components (or the process steps) may differ depending upon the manner in which the present embodiments are programmed.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

1. A method for estimating a digital twin model for decision support in a medical system, the method comprising:
acquiring measurements from a patient;
determining an estimate of a clinical biomarker from a model of organ function individualized to the patient by input of the measurements, the model of organ function having been trained for the clinical biomarker; and
displaying an image of the estimate of the clinical biomarker.

2. The method of claim 1 wherein acquiring comprises acquiring medical image data and non-medical image data for the patient.

3. The method of claim 1 wherein determining comprises determining values of parameters of the model of organ function and then determining the estimate using the model of organ function with the values of the parameters.

4. The method of claim 3 wherein the model of organ function was trained to optimize for the clinical biomarker through selection of the model of organ function from a set of multiple models based on clinical outcome prediction accuracy.

5. The method of claim 3 wherein the model of organ function is based on the optimization for the clinical biomarker through a machine-learned model relating the measurements to the values of the parameters, the machine-learned model having been trained with a loss function including a first term for a difference of training measures to model output and a second term for a difference from training biomarkers to model biomarker.

6. The method of claim 5 wherein the machine-learned model comprises a neural network.

7. The method of claim 6 wherein the machine-learned model comprises an encoder, a decoder, and an estimation network receiving values of bottleneck features between the encoder and the decoder.

8. The method of claim 1 wherein determining comprises determining the estimate of the clinical biomarker from input of the measurements to a machine learned model, which outputs the estimate, the machine-learned model having been trained based on the optimization for clinical biomarker.

9. The method of claim 8 wherein the machine-learned model was trained with a loss function with a first term for a distance between a constitutive model and a model output of the machine-learned model and a second term for a distance between biomarkers.

10. The method of claim 8 wherein the machine-learned model was trained as a forward model.

11. The method of claim 10 wherein the machine-learned model was pre-trained based on output from a generative computational model.

12. A medical system for estimating a digital twin model, the medical system comprising:
a medical imager configured to scan a patient;
an image processor configured to predict a clinical biomarker with a digital twin model of a physiological system of the patient, the digital twin model individualized to the patient based on biomarker prediction and data from the scan; and
a display configured to display the clinical outcome.

13. The medical system of claim 12 wherein the digital twin model is individualized based on the biomarker prediction by selection from a group of models where the selection was based on comparison using a plurality of samples of scan data and values of biomarkers.

14. The medical system of claim 12 wherein the digital twin model is individualized based on the biomarker prediction by output of values of parameters of the digital twin model by a machine-learned model in response to input of the data from the scan, the machine-learned model having been trained using a loss function including a distance in predicted biomarker.

15. The medical system of claim 12 wherein the digital twin model is individualized based on the biomarker prediction by output of the clinical biomarker by the digital twin model comprising a machine-learned model trained using samples of scan data and values of biomarkers.

16. The medical system of claim 15 wherein the machine-learned model was trained with a loss function having a first term based on distance from a constitutive model and a second term based on distance in predicted biomarker.

17. The medical system of claim 15 wherein the machine-learned model was trained as a forward model from the samples and therapy parameters.

18. A method for organ modeling to a patient in a medical system, the method comprising:
modeling an organ of a patient from measurements of the patient;
accounting for response to therapy, disease progression, and/or prognosis in the modeling of the organ of the patient; and
generating an estimate of patient outcome using the modeling.

19. The method of claim 18 wherein accounting comprises selecting a first model from a plurality of models, the selecting based on comparison of accuracy in prediction of the response to the therapy, disease progression, and/or prognosis of the models using testing data.

20. The method of claim 18 wherein accounting comprises estimating values of parameters of a model used in the modeling by a machine-learned model, the machine-learned model having been trained with a loss function including a loss term for the response to the therapy, disease progression, and/or prognosis.

21. The method of claim 18 wherein generating comprises generating the estimate with a machine-learned model and wherein accounting comprises having trained the machine-learned model with training data including the patient outcome.
